(19) United States
(12) Patent Application Publication
(10) Pub. No.: US 2020/0323694 A1
(43) Pub. Date: Oct. 15, 2020

(54) LAYERED APERTURED WOUND DRESSING, PROCESS OF MANUFACTURE AND USEFUL ARTICLES THEREOF

(71) Applicant: Mark E. Dillon, Center Valley, PA (US)

(72) Inventor: Mark E. Dillon, Center Valley, PA (US)

(73) Assignee: Bio Med Sciences, Inc., Allentown, PA (US)

(21) Appl. No.: 16/379,452

(22) Filed: Apr. 9, 2019

(51) Int. Cl. A61F 13/02; A61F 13/00; A61L 27/26; A61L 27/56; A61L 27/58; A61L 27/54; B32B 27/12; B32B 37/20

(52) U.S. Cl. CPC A61F 13/022; A61F 13/0253; A61F 13/00063; A61F 13/0289; A61L 27/26; A61L 27/56; B32B 2255/10; A61L 27/54; B32B 27/12; B32B 37/203; A61L 2300/404; B32B 2535/00; B32B 2255/26; A61L 27/58

(57) ABSTRACT

This invention relates to a novel wound dressing design. Particularly, this invention relates to a wound dressing which incorporates multiple distinct layers, each providing useful features and together providing a novel method of managing a variety of wound types. Moist healing, exudate management, ease of use and patient comfort are provided. In a preferred embodiment, the new dressing comprises a thin layer of gel continuously coated onto a thin film material which is laminated to an apertured mesh. Preferably, the gel coated thin film material is fenestrated or perforated. This construction improves dressing fixation, provides a semi-occlusive wound environment while simultaneously managing copious amounts of exudate.

### BACKGROUND OF THE INVENTION

### 1. Field of the Invention

This invention relates to a novel wound dressing design. Particularly, this invention relates to a wound dressing which incorporates multiple distinct layers, each providing useful features and together providing a novel method of managing a variety of wound types. Moist healing, exudate management, ease of use and patient comfort are provided.

### 2. Description of the Prior Art

In the field of wound care there exist several general categories of commonly used dressings, each with its own unique set of advantages and disadvantages. Each is indicated for certain wound conditions and user preferences. For example, conventional gauze is inexpensive and widely available, but tends to integrate into the wound as eschar forms in the wound bed and healing progresses. As a result, dressing changes can be painful and counterproductive. Hydrogel and hydrocolloid dressings are soft and gentle on the wound but are bulky and can sometimes cause tissue maceration due to excessive moisture accumulation resulting from poor exudate management. Semi-occlusive, thin polymer films coated with a pressure sensitive adhesive (“PSA”) are readily available and provide a moist healing environment with some level of exudate management. A common example of this type of dressing is a polyurethane thin film coated with an acrylic PSA. These dressings are easily applied and maintained, but can aggressively adhere to adjacent tissue complicating removal and potentially causing irritation due to the PSA.

Woven or nonwoven meshes and various types of apertured films or nettings are likewise used for a wide variety of wound dressing designs. The mesh may serve as a wound contacting surface and/or as a mechanical reinforcing mechanism for handling purposes.

Impregnated mesh dressings may use a multitude of apertured materials including but not limited to woven monofilament structures, nonwoven spunlace webs, extruded apertured materials (“scrims”), knitted textiles and even 3D printed structures, collectively “apertured meshes.” These types of dressings are easy to handle and fix in place. Yet while open porosity is helpful for exudate management, it can be problematic due to integration into the wound bed—or in extreme cases wound desiccation.

In more recent years silicones have been increasingly utilized for wound care applications, particularly for “gentle” skin adhesion. Examples include silicone impregnated into various porous substrates or coated onto thin films.

Bio Med Sciences, Inc. of Allentown, Pa. manufactures and markets the Rylon® brand of wound dressings comprised of woven polyester monofilament mesh impregnated or coated with a tacky silicone gel on one or both sides (Rylon-1 or Rylon-2 respectively). The gel is partially impregnated into the mesh so that a portion of the apertures remain open for exudate management. The mesh further provides a reinforcing mechanism sufficient enough for the retention of surgical staples when required. While these products are easy to handle and manage copious amounts of exudate, they do not typically provide a semi-occlusive environment for moist healing.

Prior art further includes silicone coated thin films that provide a semi-occlusive environment and a non-adherent wound contacting surface. While providing moist healing, these types of dressings tend to wrinkle or slip on the wound making stability and fixation problematic. An example of such a dressing is described in U.S. Pat. No. 4,832,009, which is incorporated herein by reference, and which discloses a dressing made from an interpenetrating polymer network (“IPN”) of polytetrafluoroethylene (“PTFE”) and silicone, and is presently marketed by Bio Med Sciences, Inc. as Silon-TSR® Temporary Skin Replacement. An IPN is a type of polymer/polymer composite wherein each polymer forms a continuous matrix which mutually interpenetrates the other.

As with wound dressings, there are a wide variety of wound types. Wounds can be categorized as chronic or acute. Examples of chronic wounds include venous stasis ulcers, decubitus ulcers and diabetic ulcers. Examples of acute wounds include burns, skin graft donor sites, skin graft recipient sites, abrasions and the like. Wounds are also either wide surface area or linear in nature. Wide surface area wounds such as burns are particularly problematic in comparison to linear wounds such as incisions or lacerations. With linear wounds, the tissue edges are in close proximity so there is less wound to be bridged—each side of the injured tissue is held in direct contact with the other by use of tape, sutures or staples. With wide area wounds, healing must occur from the wound bed upward. With deep wounds where the dermis is damaged or destroyed, skin grafting is required. Whether grafted or not, wide area healing is a slow, painful process.

Not only do the features required for the proper performance of a wound dressing depend on the wound type, the location on the body can have a major impact as well. The issue is particularly challenging for skin graft sites on the back or buttocks of a patient, where ordinary movement and contact with bedding can easily displace a dressing. In the case of a graft recipient site, the graft itself can even be dislodged. Similarly, chronic wounds are a challenge because they tend to produce copious amounts of exudate which often precludes the use of semi-occlusive films. This is particularly problematic for decubitus ulcers in the sacral region.

Even the same wound may require different dressings at different stages of the healing process. A venous stasis ulcer will produce copious amounts of exudate in the early stages of healing. Hydrocolloid dressings are often used on these wounds because of their high absorption capabilities. But as wounds of these type heal, the fragile epithelium can easily be damaged during dressing changes; so a non-adherent dressing may be substituted later in the healing process.

Since infection is a constant threat and potentially serious complication for any wound condition, various antimicrobial agents are used in combination or incorporated into a wide variety of wound dressings in the field. Commonly used antimicrobials include bacitracin, neomycin and polymyxin. Additionally, silver-based compounds and dressings containing silver-based compounds have become commonplace in wound care. Silver-based compounds which contain high valence states of silver (Ag2+ and Ag3+) are preferred. Furthermore, non-leaching polymeric antimicrobial agents composed of polyquat salts such as 3-methoxysilylpropyldimethyloctadecyl ammonium chloride have been used to inhibit microbial colonization.

For the reasons elucidated above no one dressing is a panacea for all wound types, circumstances or phases of healing.

In the field of polymer films, in addition to polyurethane as previously mentioned, materials such as polyethylene, polyester, polycaprolactone, vinyl and other materials including copolymers and composites (collectively “polymeric films”) are used. These polymeric films may or may not be porous or microporous. An example of such a polymeric film is described in U.S. Pat. No. 4,945,125, which is incorporated herein by reference, and which discloses a microporous polymeric IPN membrane of PTFE and silicone and is of particular interest to the present invention. For purposes of this specification, the terms “film” and “membrane” may be used interchangeably, with the term “web” applying to lengths of membranes or films that are manufactured in a continuous process of producing roll goods of such materials.

In the field of adhesives a plethora of chemical systems exist, including acrylics, hydrogels and silicones (collectively “surface adhesives.” For clarity, surface adhesives are not to be confused with contact adhesives such as cyanoacrylate glues.

In the field of silicone chemistry, there is likewise a plethora of systems known in the art. Of particular interest to the present invention are polysiloxane formulations, and in particular platinum catalyzed polydimethylsiloxane systems which are common to the medical field. In general, these formulations are two-part systems where one component contains a crosslinking agent and the other a catalyst. The two components are mixed together in liquid form. Crosslinking between polymer chains (usually accelerated with heat) causes the polysiloxane to cure or vulcanize and form a cohesive solid which can range in character from rigid elastomers to soft & pliable gels depending largely on crosslink density. Elastomers tend to have nonadherent surfaces and high durometer values while gels tend to have low durometer values and are adhesive or tacky to the touch. Such low crosslink density formulations find utility in the wound care field as “gentle adhesives” being semi-adhesive in that their tacky nature clings to the skin or wound surface but does not aggressively adhere to the point of causing wound disruption upon removal. For the purposes of this invention we shall call these types of polymer formulations (whether silicone-based or otherwise) “gels.” An example of a suitable elastomeric silicone is Dow Corning (Midland, Mich.) product code MDX4-4210. An example of a suitable silicone gel is Dow Corning product code 7-9700.

### SUMMARY OF THE INVENTION

In an effort to improve the art, I have created a dressing with a unique layered design which mitigates the problematic characteristics of silicone thin films and silicone impregnated meshes while leveraging their positive attributes.

In a preferred embodiment, the new inventive dressing comprises an IPN/gel film, which is preferably fenestrated or perforated, laminated to a silicone impregnated apertured mesh, preferably a woven mesh coated on one side (coating the second side would be superfluous in this case). For the purposes of this invention the terms “partially impregnated” and “coated” can be used interchangeably to describe the application of a polymer to an apertured mesh regardless if the polymer actually penetrates into the mesh or simply is bound to its surface. Preferably, the IPN/gel film is fenestrated or perforated, and comprises a thin layer of silicone gel continuously coated onto a silicone/PTFE IPN membrane. (For the purposes hereof, the terms fenestrated and perforated may be used interchangeably.) Due to geometry and the relative frequency of hole patterns in each layer, open holes are created in defined patterns through the entire dressing. This construction improves handling and fixation but also provides a semi-occlusive wound environment capable of managing copious amounts of exudate.

By applying the new dressing to the wound site with the gel surface of the IPN/gel membrane against the wound, the non-adherent/non-integrating advantages of the Silon-TSR dressings are preserved. At the same time, however, the handing and fixation advantages of Rylon dressings are maintained.

Fenestrations or perforations are cut through the IPN/gel material and the apertured mesh is substantially open, thereby allowing wound exudate to freely migrate from the wound through the dressing and optionally into a secondary dressing. By controlling the geometry and design of the fenestrations in the IPN/gel film relative to the apertures and holes of the impregnated mesh, a balance between moist healing and exudate management can be tailored. Consider that a large number of small holes may result in different wound management features than a small number of large holes—even if the hole area is equal. Varying the fenestration and aperture patterns of the layers of the dressings of this invention provides the ability to engineer a number of dressing designs of clinical significance. The combination of fenestrations and apertures is additive when openings align and provide a pathway for exudate to flow. Conversely it is subtractive when they do not align and moisture is retained. Given the nature of repetitive patterns superimposed upon one another, a sort of harmonic beat design occurs. Minor adjustments in one pattern or another greatly influence the overall pattern of openings that extend through the dressing. As long as the relative spacing of fenestrations to apertures is not a whole number multiple of one to the other, there is no need to register the two main layers of the dressing with each other. In other words, if the holes in the wound contacting layer exactly matched the spacing of the apertures in the mesh layer, one would have to position the two layers precisely so that openings through the combined layers are maintained. If the spacing of the holes to apertures is anything other than a whole number multiple of one to the other, every so many repeats of the patterns will cause a hole to align with an aperture. In this fashion the dressing may be engineered to provide a desired flow rate of exudate passage (e.g., a low, medium or high).

Thus, with the present invention the relative geometry of film fenestrations to mesh apertures affords a great ability to tailor exudate management versus moist healing characteristics while also providing non-adherent properties and good handling features.

### BRIEF DESCRIPTION OF THE DRAWINGS

FIG. 1a shows a cross-sectional view of a preferred embodiment of the invention. FIG. 1b shows a cross-sectional view of an alternative preferred embodiment of the invention.

FIG. 2a shows a plan view of a dressing (60) of this invention viewed from the wound contacting side, illustrating a preferred fenestration or perforation pattern.

FIGS. 2b, 2c, and 2d show plan views of the dressing (60) of this invention viewed from the wound contacting side, illustrating alternative fenestration or perforation patterns.

FIG. 3a shows a plan view of an apertured woven mesh material suitable for use for the apertured mesh layer (50) of the invention. FIG. 3b shows a plan view of an aperture mesh layer (50) partially coated with a silicone polymer (110).

FIG. 4 shows a photographic plan view of the inventive dressing (60) viewed from the side of the dressing (60) that faces towards the wound application site when the dressing (60) is applied to the wound application site.

FIG. 5a is a simplified schematic diagram of a preferred manufacturing process used to produce the IPN/gel web (280) of the invention. FIG. 5b is a simplified schematic diagram of a preferred manufacturing process used to produce the impregnated mesh web (340) (that is, apertured mesh layer (50) at least partially impregnated with silicone (40)) of this invention.

FIG. 6a is a simplified schematic diagram of a preferred manufacturing process for laminating the two webs (that is, the IPN/gel layer (15) and the impregnated mesh layer (45)) together, placing them onto a suitable release liner (260) thereby creating construction (370), and die cutting shapes (410) therefrom.

FIG. 6b is an enlarged view of a portion shown in the dashed circle A of FIG. 6a.

### DETAILED DESCRIPTION OF PREFERRED EMBODIMENTS

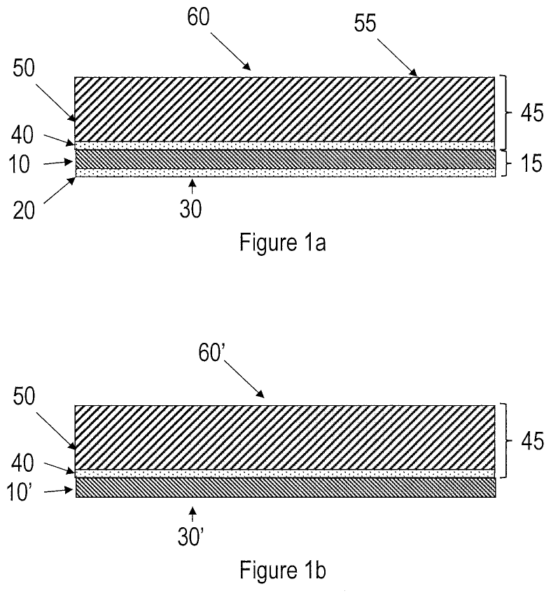

FIG. 1 shows a cross-sectional view of a preferred embodiment of the invention. As shown in FIG. 1, the inventive dressing (60) comprises a semi-occlusive polymeric membrane layer (10)(such as a silicone/PTFE IPN membrane) that is coated with a tacky silicone gel (20) on one side, and that on its other side is bonded to the silicone coated surface (40) of an apertured mesh layer (50). The silicone gel (20) provides the wound dressing (60) with a gently adhesive wound contacting surface (30). The wound contacting surface (30) of the dressing (60) faces the wound application site and comes into contact with the wound when the dressing (60) is applied to the wound application site, and the surface (55) of the apertured mesh layer (50) faces away from the wound application site when the dressing (60) is applied to the wound application site. Due to the gently adhesive characteristic of the silicone gel (20), the wound dressing (60) easily peels off a wound when desired without the wound contacting surface (30) integrating with the wound.

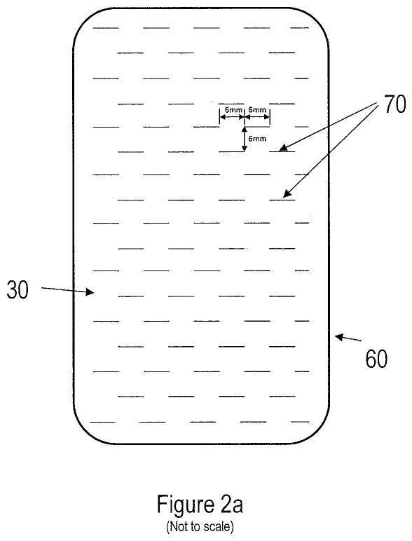

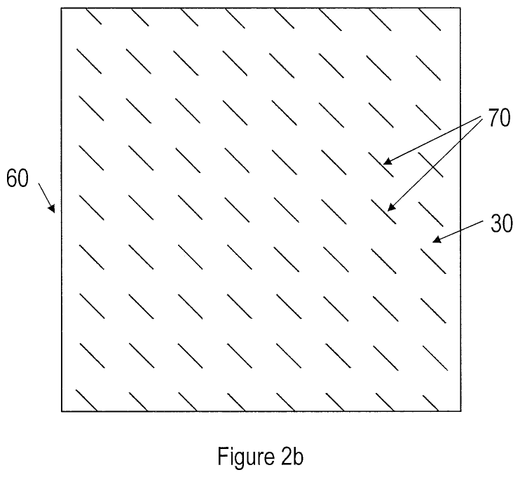

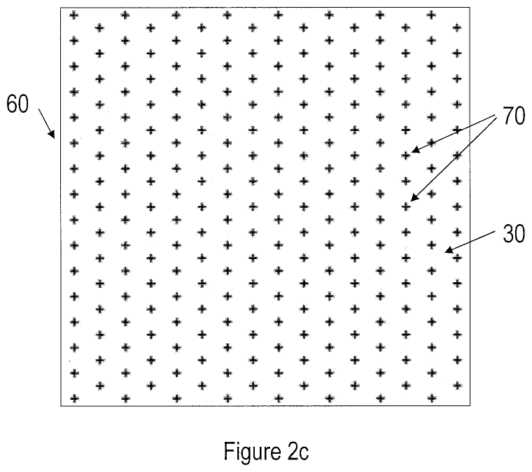

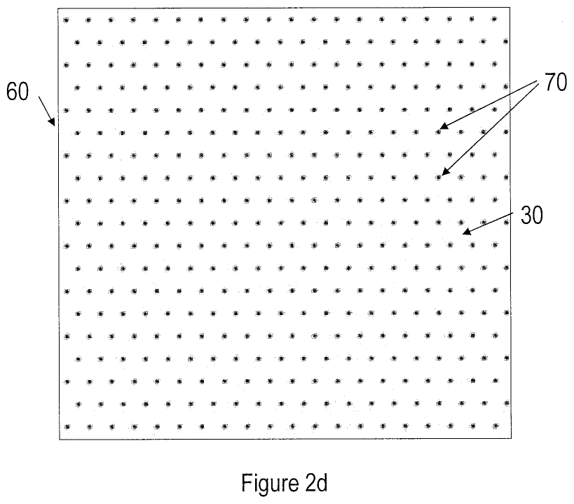

FIG. 2a shows a plan view of a dressing (60) of this invention viewed from the wound contacting side, illustrating a preferred fenestration or perforation pattern. Fenestrations (70) are cut through the IPN/gel film but not through the apertured mesh. FIGS. 2b, 2c, and 2d show plan views of the dressing (60) of this invention viewed from the wound contacting side, illustrating alternative fenestration or perforation patterns. Many variations of the fenestration or perforation patterns other than those shown in FIGS. 2a, 2b, 2c and 2d are possible. It should be noted that the final shape and dimensions of the opening created by the fenestration tooling may not match precisely the dimensions of the tooling design, as tension applied to the film layer downstream from the fenestration process may affect the final geometry. For example, a slit fenestration may become an oblong oval hole if tension is applied perpendicular to the slit during later processing.

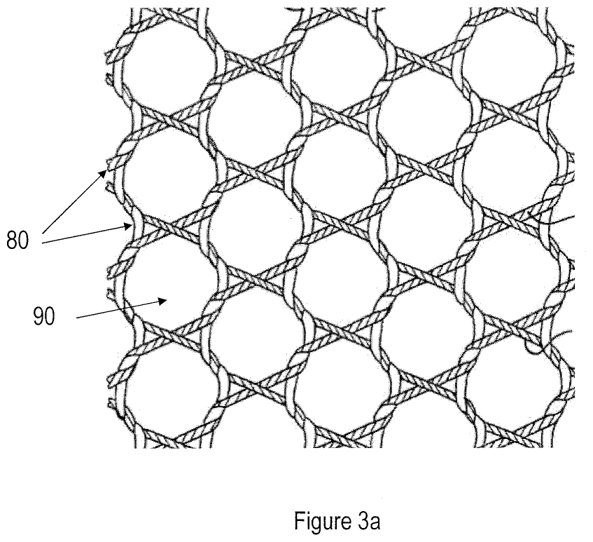

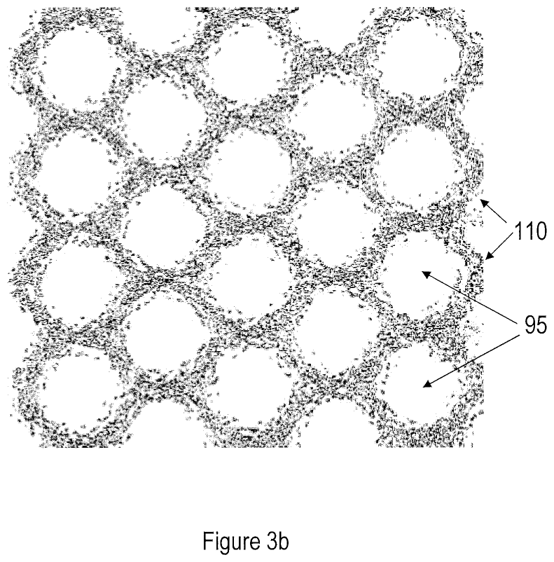

FIG. 3a shows a plan view of an apertured woven mesh material suitable for use for the aperture mesh layer (50) of the invention. Monofilaments (80) define an open aperture structure (90). FIG. 3b shows a plan view of the apertured woven mesh of FIG. 3a partially coated with silicone (110) leaving openings (95) in the apertured mesh.

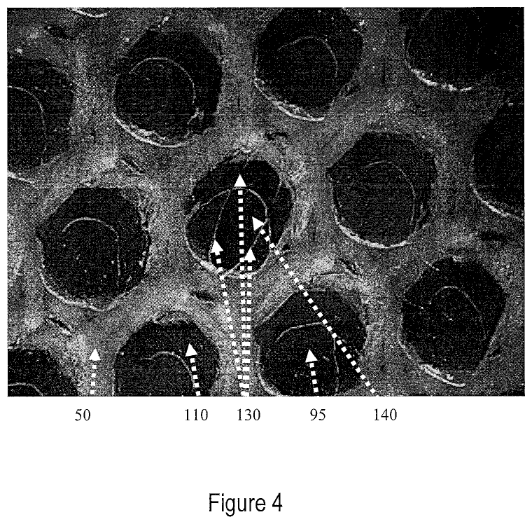

FIG. 4 shows a photographic plan view of the inventive dressing (60) viewed from the side of the dressing (60) that faces towards the wound application site when the dressing (60) is applied to the wound application site, in which an apertured woven mesh (50) has been partially impregnated with silicone (110) in such a fashion as to maintain apertures (95) of the mesh (50) at least partially open, and in which the fenestration pattern (70) of the IPN/gel film (15) is present (not visible in image). The opening created by the fenestration pattern is defined by the edge of the IPG/gel film as defined by the circumscribed points 130 (slightly visible in the image). It should be noted that visualization of the IPN/gel (15) and openings defined by circumscribed points (130) is difficult due to the highly transparent nature of the INP/gel film (15). In this fashion the fenestration holes in the IPN/gel film (15) leave openings (140) penetrating entirely through the dressing. In other words, where a fenestration hole defined by circumscribed points (130) aligns with an aperture hole (95) a pathway (140) for exudate migration is created. Everywhere else is occluded by the surface (30) of the IPN/gel film 15.

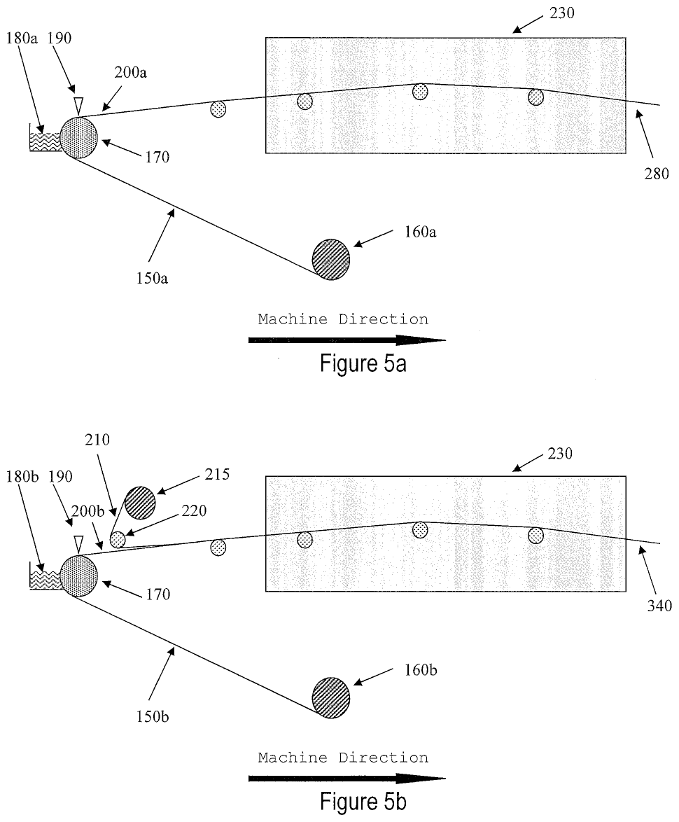

FIG. 5a is a simplified schematic diagram of a preferred manufacturing process used to produce a web (280) of the IPN/gel film (15) on a polypropylene coated paper carrier substrate (150a) of the invention. A PTFE/silicone IPN (10) on carrier substrate (150a) web is unwound from a roll (160a) and passed over a roller (170). The IPN on carrier substrate (150a) passes through a reservoir of uncured liquid silicone (180a) and an adjustable blade “knife” (190) is set to meter off excess liquid silicone (180a) leaving behind the desired thickness of silicone gel (200a) on the IPN (10) on carrier substrate (150a). Preferably, but optionally, the uncured liquid silicone (180a) may contain an antimicrobial substance, such as 3% by weight of a non-leaching polyquat antimicrobial (3-trihydroxysilylpropyldimethyloctadecyl ammonium chloride) or 3% by weight of silver oxysalts. The resultant construction (200a) of uncured gel (20) on IPN (10) with carrier substrate (150a) then is passed through a tunnel style oven (230) to apply heat and effectuate crosslinking of the silicone to form a web (280) of IPN/gel film (15) on carrier substrate (150a). The web (280) of IPN/gel film (15) on carrier substrate (150a) is wound onto a master roll (290).

FIG. 5b is a simplified schematic diagram of a preferred manufacturing process used to produce a web (340) of the at least partially impregnated apertured mesh material (45) (that is, a web of the apertured mesh material (50) at least partially impregnated with silicone (110) and having a silicone coated surface (40)) of this invention. A polypropylene coated paper carrier substrate (150b) is unwound from a roll (160b) and passed over a roller (170). The carrier substrate (150b) passes through a reservoir of uncured liquid silicone (180b) and an adjustable blade “knife” (190) is set to meter off excess liquid silicone (180b) (200b, 40) on the carrier substrate (150b). Preferably, but optionally, the uncured liquid silicone leaving behind the desired thickness of silicone gel (180b) may contain an antimicrobial substance, such as 3% by weight of a non-leaching polyquat antimicrobial (3-trihydroxysilylpropyldimethyloctadecyl ammonium chloride) or 3% by weight of silver oxysalts. An apertured mesh (210, 50) is unwound from a roll (215) and passed over a “lay down” roller (220) to be put into contact with the uncured silicone (200b, 40) on the carrier substrate (150b). The resultant material then is passed through a tunnel style oven (230) to apply heat and effectuate crosslinking of the silicone to form a web (340) of at least partially impregnated apertured mesh material (45) on carrier substrate 150b, and the resultant web (340) is then taken-up onto (wound onto) a new master roll (355).

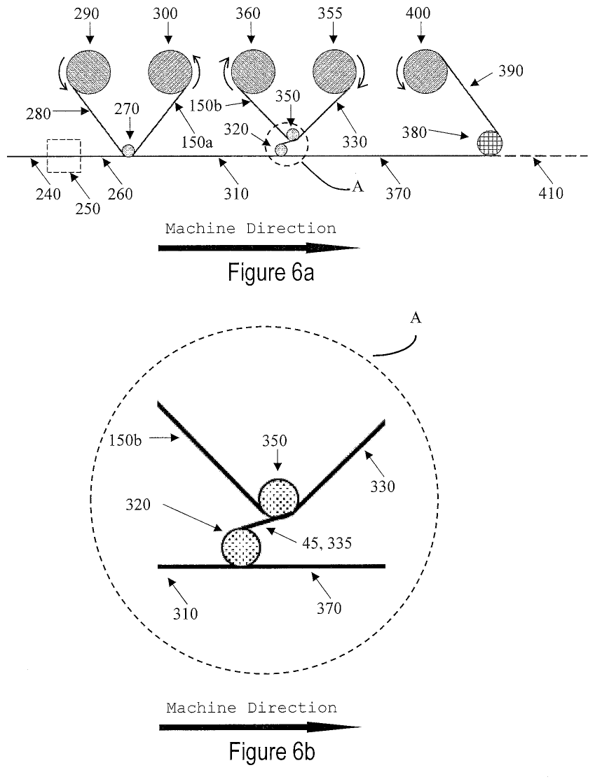

FIG. 6a is a simplified schematic diagram of a preferred manufacturing process for laminating the two webs (280) and (340) (that is, the web (280) of the IPN/gel film (15) and the web (340) of at least partially impregnated apertured mesh material (45) together, placing them onto a suitable release liner, and die cutting shapes therefrom. FIG. 6b is an enlarged view of a portion of FIG. 6a (the dashed circle labeled “A.” A release liner (240), such as polypropylene coated paper or polyester film, is passed through a splitting station (dashed box 250) that creates slit liner (260) having a butterfly fold, score or other suitable means (“slit”) of facilitating final removal and application of the finished dressing. The slit liner (260) is passed under an idler roller (270). A web (280) of IPN/gel film (15) positioned on the carrier substrate (150a) is unwound from a master roll (290) with the gel side facing the slit liner (260). The carrier substrate (150a) attached to the web (280) of the IPN/gel film (15) in the manufacturing process of FIG. 5a for making the web (280) is now removed from IPN/gel film (15) and rewound onto a roll (300) to be discarded or preferably recycled. The web (280) of IPN/gel film (15) is passed around idler roller (270) to meet the slit liner (260). Prior to the web (280) of IPN/gel film (15) passing around idler roller (270), optionally, but preferably, the web (280) of IPN/gel film (15) may be passed through a suitable cutting tool to create fenestrations (70) through the IPN/gel film (15). The optionally fenestrated web of the IPN/gel film (15) is thus put in contact with the slit release liner (260) so that the gel side contacts the slit release liner (260) and the IPN (10) side is facing away from the slit release liner. The IPN/gel film (15) on slit liner (260) (together identified in FIGS. 6a and 6b with the reference number (310)) is then passed under a second idler roller (320).

A web (340) of at least partially impregnated apertured mesh material (that is, the web (340) comprising a web (45) of apertured mesh material (50) at least partially impregnated with silicone (110) and having a silicone coated surface (40)) on carrier substrate (150b) (together identified in FIGS. 6a and 6b with the reference number (330)) is unwound from a master roll (355) with the mesh (50) side facing outwardly away from the roll (355) and the carrier substrate (150b) contacting idler roller (350). The backing carrier substrate (150b) is removed and rewound onto a roll (360) to be discarded or preferably recycled. The process of removing the carrier substrate (150b) from the silicone surface (40) creates holes (95) where the silicone is not supported by apertured matrix (50, 80). The free-standing web (335, 45) of at least partially impregnated apertured mesh material is passed under idler roller (350) and around idler roller (320) to meet web (310) (that is the IPN side (10) of IPN/gel film (15) on slit liner (260). Referring to FIG. 6b, the at least partially coated apertured mesh web material (that is, the free-standing web (335, 45) of at least partially impregnated apertured mesh material used to form the layers of the wound dressing (60) comprising the apertured mesh layer (50) at least partially impregnated with silicone (110) and having a silicone coated surface (40), and identified in FIGS. 1a and 1b with reference number (45) and in FIG. 6b additionally as 335 is passed around roller 320, and the coated side (that is, the side with the silicone coated surface (40)) of the coated apertured mesh web material is put in contact with the IPN side (10) of the IPN/gel film (15) of web (310) positioned on slit liner (260) resulting in construction (370). Preferably, a pressure applying nip roller is used at the lamination point to cause the two layers to firmly adhere together (not shown). It should be noted that in the described process holes (95) are created when the carrier substrate (150b) is removed from the web (340) of the at least partially impregnated apertured mesh material, as free-standing silicone (200b) that is not supported by the matrix (e.g., the monofilaments 80) of the apertured mesh 50 inherently adheres to carrier substrate (150b) resulting in the openings (95) in the at least partially impregnated apertured mesh (45, 335).

The constructed material (370) of layered apertured web (45) bound to the IPN side (10) of layer (15) on slit release liner (260) is then passed through a die cutting apparatus (380) to punch-cut the final wound dressing shapes. The remaining matrix not cut from web (370) (that is, the remaining material (390) left behind from the cutting process) is then rewound onto a roll (400) to be discarded or preferably recycled. The individual dressings (60) (identified in FIG. 6a with reference number (410)) resulting from these steps are then packaged and sterilized according to established methods.

Turning now to FIG. 1b, there is shown a preferred alternative embodiment of the invention. Here, the inventive would dressing (60′) is substantially the same as wound dressing (60), except instead of having a semi-occlusive polymeric membrane layer such as an IPN thin film (10) coated with a silicone gel (20), wound dressing (60′) is provided with a semi-occlusive polymeric membrane layer such as an IPN thin film (10′) that is not coated with a silicone gel (20), since the thin film (10′) has been formulated to be itself semi-adherent and therefore have a tacky gentle adhesive wound contacting surface (30′). Accordingly, like the gently adhesive wound contacting surface (30) of the wound dressing (60), the tacky thin film (10′) enables the wound dressing (60′) to easily peel off a wound when desired without its wound contacting surface (30′) integrating with the wound, and like the tacky wound contacting surface (30) of the wound dressing (60), the low tack wound contacting surface (30′) of the wound dressing (60′) helps the wound dressing stay in place on a wound but substantially does not permanently adhere to the wound and is substantially non-integrating with the wound. Preferably, the thin film (10′) may be a silicone/PTFE IPN membrane formulated to be semi-adherent (that is, to have a gentle adhesive surface). Preferably, but optionally, the thin film (10′) may be fenestrated. The wound dressing (60′) may be made in the same manner as the wound dressing (60), except the application of silicone gel layer (20) may be omitted. Further, the wound dressing (60′) may be used in the same manner as wound dressing (60), except rather than placing the wound contacting surface (30) of wound dressing (60) into contact with a wound as would be done with wound dressing (60), the wound contacting surface (30′) of wound dressing (60′) is placed into contact with a wound.

As with dressing (60), dressing (60′) may be engineered to provide a desired flow rate (e.g., a low, medium or high) rate of exudate passage.

A preferred embodiment of this invention includes fenestrations through the wound contacting polymer film layer, however it is contemplated that the use of porous or microporous polymeric films may be utilized so that fenestrations or perforations are not necessary to achieve the same basic function of the present invention.

The following examples are not intended to be limiting, as variations on these designs, configurations and processes would be obvious to those skilled in the art. It is obvious that the relative layers of the dressing of this invention may be substantially varied. Example 1 shows an IPN/gel film of approximately 40 microns thickness, but at between 10 and 200 microns is sufficient. Example 1 also shows a woven mesh of 380 microns in thickness with a final partially impregnated apertured mesh of approximately 420 microns; however, these layers may range from 100 microns 600 microns in combination or independently.

Likewise, it is believed that other materials could be used to achieve the same dressing design. Throughout this specification the use of fenestrations of the polymer film layer is described as a preferred embodiment; however, microporous thin films, particularly those that resorb or dissolve also may be used regardless of being fenestrated or not.

Finally, in addition to the cut dressing shapes described herein, a useful alternative is to provide small rolls of the inventive material without a release liner for “tape-like” or circumferential wrap style application.

### Example 1

A continuous web of polydimethylsiloxane and polytetrafluoroethylene IPN was manufactured according to established methods on a suitable carrier substrate, and then coated with a silicone gel using the equipment and process shown and described in connection with FIG. 5a. The IPN/gel film produced measured approximately 40 microns in thickness and was subsequently passed through a tool to create fenestrations substantially as shown in FIG. 2a.

A web of woven mesh approximately 380 microns thick was manufactured according to established methods, and then partially impregnated with silicone gel on a suitable carrier substrate using the equipment and process shown and described in connection with FIG. 5b resulting in a finished construction of approximately 420 microns in thickness.

Using the equipment and process shown and described in connection with FIGS. 6a and 6b, layered apertured dressings were created on a butterfly folded polypropylene coated paper release liner, cut into 13×25 cm sheets, then packaged and sterilized for final use.

### Example 2

Using bench-top analogs of the processes described in connection with FIGS. 5a, 5b, 6a and 6b, Example 1 was repeated, except a nonwoven mesh of approximately 325 microns in thickness was used instead of the woven mesh. The nonwoven mesh was spunlace polyester with apertures in an isotropic square pattern of 6 holes per linear cm. The finished construction measured approximately 365 microns in thickness.

### Example 3

Examples 1 and 2 are repeated, except a silicone gel containing 3% by weight of a non-leaching, polyquat antimicrobial (3-trihydroxysilylpropyldimethyloctadecyl ammonium chloride) is used. That is, the uncured liquid silicone (180a) in the reservoir shown in FIG. 5a contains 3% by weight of a non-leaching, polyquat antimicrobial (3-trihydroxysilylpropyldimethyloctadecyl ammonium chloride).

### Example 4

Examples 1, 2 and 3 are repeated, except a silicone gel containing 3% by weight of silver oxysalts is used. That is, the uncured liquid silicone (180a) in the reservoir shown in FIG. 5a contains 3% by weight of silver oxysalts.

### Example 5

Examples 1 through 4 are repeated except that a silicone gel containing 3% by weight of silver oxysalts is used. That is, the uncured liquid silicone (180b) in the reservoir shown in FIG. 5b contains 3% by weight of silver oxysalts.

### Example 6

Examples 1 through 5 are repeated except a silicone gel containing 3% by weight of a non-leaching, polyquat antimicrobial (3-trihydroxysilylpropyldimethyloctadecyl ammonium chloride) is used. That is, the uncured liquid silicone (180b) in the reservoir shown in FIG. 5b contains 3% by weight of a non-leaching, polyquat antimicrobial (3-trihydroxysilylpropyldimethyloctadecyl ammonium chloride).

### Example 7

Examples 1 through 6 are repeated, except a PTFE/silicone IPN (10′) is formulated using a silicone gel so that wound contacting surface (30′) is inherently tacky and except the step of coating the IPN with a silicone gel using the equipment and process shown and describe in connection with FIG. 5a is omitted.

### Example 8

Example 7 are is repeated, except a microporous resorbable polymeric membrane made of a copolymer of polylactic acid, polylactide, trimethylene carbonate, e-caprolactone is used instead of a PTFE/silicone IPN film (10′) so that the wound contacting surface (30′) is semi-adherent by means of microporous capillary action and except curing is done by passing through the oven (230) at low temperature due to temperature sensitivity of the copolymer.

Preferably, in accordance with the invention, a method of managing a variety of wound types comprises the steps of providing a wound dressing of the invention, in which the wound dressing comprises multiple layers, wherein a first wound contacting layer is fenestrated or perforated semi-occlusive thin film membrane which is a semi-adhesive gel or other polymeric formulation that is inherently tacky, a second more distal layer is a non-continuous silicone gel partially penetrating or otherwise adhering to an apertured mesh, and applying the wound dressing to a wound with the first wound contacting layer being against the wound, so that wound exudate passes through the wound dressing, while integration of the wound dressing into the wound and slippage and wrinkling of the wound dressing on the wound are limited. In this embodiment, the first wound contacting layer of the wound dressing, as well as the non-continuous silicone gel partially penetrating or otherwise adhering to an apertured mesh may include an antimicrobial substance, such as 3% by weight of a non-leaching polyquat antimicrobial (for example, 3-trihydroxysilylpropyldimethyloctadecyl ammonium chloride) or 3% by weight of silver oxysalts.

In accordance with the invention, a method of managing a wound comprises providing a wound dressing of the invention, in which the wound dressing comprises multiple layers, wherein a first wound contacting layer is a semi-adhesive gel or other polymeric film, a second more distal layer is an apertured mesh thus providing a moist healing environment for the wound, while (a) limiting slippage and wrinkling of the wound dressing on the wound, (b) permitting wound exudate to pass through the wound dressing, and (c) limiting integration of the wound dressing into the wound. In this embodiment, the first wound contacting layer of the wound dressing, as well as the silicone gel of the at least partially impregnated apertured mesh may include an antimicrobial substance, such as 3% by weight of a non-leaching polyquat antimicrobial (for example, 3-trihydroxysilylpropyldimethyloctadecyl ammonium chloride) or 3% by weight of silver oxysalts.

The apertured mesh may be woven or knitted textile material, or a nonwoven material, or an extruded scrim.

Preferably, in accordance with the invention, a method of manufacturing a multilayered wound dressing also comprises the steps of (1) creating a thin film with a suitable wound contacting gentle adhesive surface (that is, creating a thin film with a suitable wound contacting low tack surface), (2) creating an at least partially impregnated apertured mesh by at least partially impregnating an apertured mesh with a polymer gel so that apertures of the at least partially impregnated apertured mesh remain open to the passage of wound exudate, the apertured mesh having an outer surface that faces away from a wound application site, and (3) adhering the thin film and the at least partially impregnated apertured mesh together creating a multilayered wound dressing having a wound contacting surface and a distal surface, the wound contacting surface being the wound contacting gentle adhesive surface (that is, the wound contacting tacky surface) of the thin film and the distal surface being the outer surface of the apertured mesh. In a preferred embodiment of the method of manufacturing the multilayered wound dressing, the method may include a step of fenestrating the thin film to create openings for exudate passage. The thin film may be, for example, a semi-occlusive polymeric membrane such as a silicone/PTFE IPN membrane formulated to be inherently tacky and therefore have a gentle adhesive surface.

Having thus described my invention, I claim:

1. A wound dressing comprising of multiple layers, wherein a first wound contacting layer is a semi-adherent polymer, a second more distal layer is a semi-occlusive thin film membrane, and a third more distal layer is a non-continuous silicone gel partially penetrating or otherwise adhering to an apertured mesh.

2. The wound dressing of claim 1, wherein the apertured mesh is a woven or knitted textile material.

3. The wound dressing of claim 1, wherein the apertured mesh is a nonwoven material.

4. The wound dressing of claim 1, wherein the apertured mesh is an extruded scrim.

5. The wound dressing of claim 1, wherein the first wound contacting layer and the second more distal layer are fenestrated.

6. The wound dressing of claim 1, further including an antimicrobial substance contained in the first layer.

7. The wound dressing of claim 1, further including an antimicrobial substance contained in the third more distal layer of non-continuous silicone gel partially penetrating or otherwise adhering to an apertured mesh.

8. The wound dressing of claim 1, further including an antimicrobial substance contained in the first layer and an antimicrobial substance contained in the third more distal layer of non-continuous silicone gel partially penetrating or otherwise adhering to an apertured mesh.

9. A method of manufacturing a multilayered wound dressing comprising the steps of (1) creating a thin film with a suitable wound contacting semi-adherent surface, (2) creating an at least partially impregnated apertured mesh by at least partially impregnating an apertured mesh with a polymer gel so that apertures of the at least partially impregnated apertured mesh remain open to passage of wound exudate, the apertured mesh having an outer surface that faces away from a wound application site, and (3) adhering the thin film and the at least partially impregnated apertured mesh together creating a multilayered wound dressing having a wound contacting surface and a distal surface, the wound contacting surface being the wound contacting semi-adherent surface of the thin film and the distal surface being the outer surface of the apertured mesh.

10. The method of claim 9, further including fenestrating said thin film to create openings for exudate passage.

11. A method of managing a variety of wound types, comprising the steps of providing the wound dressing of claim 5, and applying the wound dressing to a wound with the first wound contacting layer being against the wound, so that wound exudate passes through the wound dressing, while integration of the wound dressing into the wound and slippage and wrinkling of the wound dressing on the wound are limited.

12. The method of claim 11, further including providing an antimicrobial substance in the first layer of the wound dressing.

13. A method of managing a wound, comprising providing the wound dressing of claim 5,
applying the wound dressing to a wound with the first wound contacting layer being against the wound, and,
creating with the wound dressing on the wound a moist healing environment for the wound, while (a) limiting slippage and wrinkling of the wound dressing on the wound, (b) permitting wound exudate to pass through the wound dressing, and (c) limiting integration of the wound dressing into the wound.

14. A method of managing a wound, comprising
providing the wound dressing of claim 1,
applying the wound dressing to the wound with the first wound contacting layer being against the wound, and
creating with the wound dressing on the wound a moist healing environment for the wound, while (a) limiting slippage and wrinkling of the wound dressing on the wound, and (b) limiting integration of the wound dressing into the wound.

15. A wound dressing, comprising
a semi-adherent wound contacting polymeric membrane layer having a first side and a second side, and
an apertured mesh layer, the apertured mesh layer having a first side and a second side, the first side of the apertured mesh layer having an outer surface, the apertured mesh layer comprising a material having a plurality of apertures extending therethrough from the first side of the apertured mesh layer to the second side of the apertured mesh layer, the apertured mesh layer being at least partially impregnated with silicone to coat at least a portion of the material comprising the apertured mesh layer forming a coating of silicone on at least a portion of the outer surface of the first side of the apertured mesh layer while keeping at least a portion of the apertures open to allow exudate to migrate through the apertured mesh layer,
the silicone at least partially impregnating the apertured mesh layer to form a coating of silicone on at least a portion of the outer surface of the first side of the apertured mesh layer being bonded to the apertured mesh layer, and
the coating of silicone on the outer surface of the first side of the apertured mesh layer being bonded to the second side of the polymeric membrane.

16. The wound dressing of claim 15, wherein the polymeric membrane layer is a semi-occlusive thin film membrane.

17. The wound dressing of claim 15, wherein the polymeric membrane layer is a silicone/PTFE IPN membrane.

18. The wound dressing of claim 15, wherein the polymeric membrane layer is a microporous resorbable polymeric membrane.

19. The wound dressing of claim 15, wherein the polymeric membrane layer is a microporous resorbable polymeric membrane comprising a copolymer of polylactic acid, polylactide, trimethylene carbonate, and e-caprolactone.

20. The wound dressing of claim 15, wherein the apertured mesh layer is a woven or knitted textile material.

21. The wound dressing of claim 15, wherein the apertured mesh layer is a non-woven material.

22. The wound dressing of claim 15, wherein the polymeric membrane layer is fenestrated.

23. A wound dressing, comprising
a polymeric membrane layer having a first side and a second side,
an apertured mesh layer, the apertured mesh layer having a first side and a second side, the first side of the apertured mesh layer having an outer surface, the apertured mesh layer comprising a material having a plurality of apertures extending therethrough from the first side of the apertured mesh layer to the second side of the apertured mesh layer, the apertured mesh layer being at least partially impregnated with silicone to coat at least a portion of the material comprising the apertured mesh layer forming a coating of silicone on at least a portion of the outer surface of the first side of the apertured mesh layer while keeping at least a portion of the apertures open to allow exudate to migrate through the apertured mesh layer, and
a silicone gel coating the first side of the polymeric membrane layer,
the silicone at least partially impregnating the apertured mesh layer to form a coating of silicone on at least a portion of the outer surface of the first side of the apertured mesh layer being bonded to the apertured mesh layer,
the coating of silicone on the outer surface of the first side of the apertured mesh layer being bonded to the second side of the polymeric membrane,
the silicone gel coating the first side of the polymeric membrane being bonded to the polymeric membrane,
the wound dressing having a wound contacting layer, the silicone gel coating the first side of the polymeric membrane being the wound contacting layer, and
the silicone gel coating the first side of the polymeric membrane layer being semi-adhesive such that the wound dressing stays in place on a wound but substantially does not permanently adhere to a wound and is substantially non-integrating with a wound.

24. The wound dressing of claim 23, wherein the polymeric membrane layer and the silicone gel coating the first side of the polymeric membrane layer are fenestrated.

25. The wound dressing of claim 23, further including an antimicrobial agent in the silicone gel coating the first side of the polymeric membrane layer.

26. The wound dressing of claim 24, further including an antimicrobial substance in the silicone gel coating the first side of the polymeric membrane layer.

27. A wound dressing comprising multiple layers, wherein a first wound contacting layer is a semi-adherent polymer film, and a second layer is a non-continuous silicone gel partially penetrating or otherwise adhering to an apertured mesh.
